(19) United States
(12) Patent Application Publication
(10) Pub. No.: US 2025/0000640 A1
(43) Pub. Date: Jan. 2, 2025

(54) DRUG DELIVERY SYSTEM AND METHODS FOR AN IMPLANTABLE MEDICAL DEVICE

(71) Applicant: Feng's Inc, Fullerton, CA (US)

(72) Inventors: TONY FENG, Fullerton, CA (US); HOANG OANH VU, Fullerton, CA (US)

(21) Appl. No.: 18/767,666

(22) Filed: Jul. 9, 2024

Related U.S. Application Data
(63) Continuation of application No. PCT/US23/36724, filed on Nov. 2, 2023.
(60) Provisional application No. 63/523,639, filed on Jun. 27, 2023.

(51) Int. Cl. A61F 2/14

(52) U.S. Cl. CPC A61F 2/148

(57) ABSTRACT

A method of manufacture is presented. The method includes providing a medical device, a drug delivery module, and an assembly device. The medical device comprises a first coupling feature; the drug delivery module comprises a second coupling feature and a product well. The assembly device comprises a top body comprising an implant press, middle body defining a module recess, and a base body defining a device receiver. The medical device is placed in the device receiver and the drug delivery module in the module recess. The top body, the middle body, and the base body, are aligned. Then a force is applied to the top body, wherein the force is sufficient for the implant press to cause the first coupling feature and the second coupling feature to couple to each other.

### CROSS-REFERENCE TO RELATED APPLICATIONS

This application claims the benefit of U.S. provisional application No. 63/523,639 entitled “Drug Delivery System and Methods for an Implantable Medical Device,” filed on Jun. 27, 2023, and PCT/US23/36724, filed on Nov. 2, 2023, the enclosures of which are incorporated entirely by reference herein.

### BACKGROUND

### Technical Field

The disclosures herein relate to a drug delivery system for an implantable medical device. Specifically, the disclosed invention relates to components of a drug-eluting device system configured to couple to an implantable medical device and methods of use for attaching the drug-eluting device to an implantable medical device immediately prior to surgical implantation.

### State of the Art

Drug delivery for the treatment of eye diseases has traditionally been by administering small-molecule therapeutics via eyedrops. More recently, intraocular injections have been employed for “back of the eye” delivery of large-molecule compositions to the vitreous body, supra-choroidal space, and sub-retinal space. These methods deliver a single dose of medication, and repeated interval dosing is generally necessary for successful treatment. The need for administering multiple doses at scheduled time intervals, however, requires patient compliance. Missed doses and incomplete courses of therapy yield suboptimal results. Patients forget to take their eyedrops or find putting in eyedrops at a scheduled time is inconvenient and disruptive. Moreover, intraocular injections cannot be performed by the patient, and they are uncomfortable. Even worse, the experience of having a needle inserted into one's eye is frightening for almost everyone, can be painful, and carries a risk of serious complications that include bleeding, retinal detachment, and infectious ophthalmitis, among others.

To improve therapeutic outcomes by reducing patient compliance requirements, intra-ocular drug-eluting implants have been developed in recent years. Such intra-ocular drug-eluting implants, however, act solely to provide a therapeutic medicament. The drug-eluting implants currently known in the art address no other clinical issue and have no other therapeutic purpose separate from providing medication proximate to a target tissue, such as the interior of the eye, for example.

Cataracts are considered a normal consequence of aging. Additionally, cataracts may be caused by or commonly be associated with other diseases of the eye, such as glaucoma, diabetes, and a history of previous eye surgery or injury, for example. According to the National Eye Institute, most people either have cataracts or have had cataract surgery by age 80. Cataracts are treated surgically. This involves removing the cataract-clouded lens and inserting an intraocular lens (“IOL”) replacement.

Many different pharmacologic therapies are commonly prescribed to treat this variety of both primary diseases of the eye and secondary ocular pathology arising from systemic illness, like diabetes, in patients who also require cataract surgery. Because cataract patients commonly have other conditions amendable to treatment with intraocular drugs and because patients requiring intraocular injections or daily eyedrops commonly have cataracts, an IOL having drug-eluting capability is desirable. IOLs used in cataract surgery are not, however, “one size fits all.” As with an external corrective lens, i.e., glasses and contact lenses, the eye surgeon selects an IOL having a refractive index, focal length, and diameter suitable for a given individual patient from the many sizes and varieties of IOLs that are currently available.

It is not practical to provide the necessary variety of specific IOL types, each type configured to deliver one each of the vast range of different medicaments for treating each different eye disease.

For at least the foregoing reasons, there is a need for a drug delivery system having an implantable medical device that can be coupled to a separate drug-eluting module, creating a practical solution to improve patient compliance for long-term treatment of chronic conditions that overcomes the deficiencies discussed herein above. An example of such a drug delivery system is a replacement IOL having an interchangeable drug-eluting mechanism wherein any number of therapeutic medications can be coupled to the IOL immediately prior to implantation, is needed.

### BRIEF SUMMARY

Disclosed herein are embodiments of a drug-delivery implantable medical device system. Example embodiments of a drug-eluting intraocular implant coupled to a drug delivery module are discussed at length; however, this is not meant to be limiting. Other implantable medical devices configured for implantation in non-ocular locations throughout the body that are coupled to a drug delivery module at the time of implantation are contemplated by the following disclosures, and alternative examples will be apparent to those of skill in the art of implantable medical devices generally.

A key feature of the example embodiments disclosed herein is the ability to treat two separate, distinct clinical conditions—a first condition treated with an implantable medical device and a second, distinct, and possibly unrelated condition with a timed-release drug-eluting composition containing a medicament loaded into a product well of a drug delivery module that is coupled to the implantable medical device at the time of the implantation procedure. The capability to deliver a therapeutic medication directly to a target tissue minimizes the amount of medication entering the patient, reduces the overall amount of medication needed, and, in many cases, is more efficacious than oral, topical, topical drops, inhaled aerosols, or other more generalized delivery methods that do not focus a concentrated, continuous, timed-release done of the therapeutic medicament proximate to the tissue target of the therapeutic medication. Two separate conditions can be treated efficiently because the drug delivery module pre-loaded with a therapeutic medicament may be selected separately from the implantable medical device, making possible dozens or even hundreds of possible combinations between different types, sizes, and configurations of the implantable medical device specific to the physical and therapeutic needs of an individual patient and the drug delivery module bearing the pharmacologic therapy selected by the patient's healthcare providers to treat the second condition.

Disclosed is a drug delivery system for an implantable medical device comprising an implantable medical device selected to deliver a first treatment for a first condition to an individual patient and a drug delivery module comprising a medicament selected to deliver a second treatment for a second condition to the individual patient, wherein the drug delivery module becomes coupled to the implantable medical device immediately prior to implantation in the patient.

In some embodiments, the implantable medical device comprises an intraocular lens. In some embodiments, the medicament is combined with a carrier composition. In some embodiments, the carrier composition is a biopolymer.

In some embodiments, the first condition is an ocular condition. In some embodiments, the first condition is a cataract. In some embodiments, the second condition is a different condition than the first condition. In some embodiments, the second condition is an ocular condition. In some embodiments, the second condition is glaucoma.

In some embodiments, the implantable medical device comprises an intraocular lens and a haptic arm coupled to the intraocular lens at a flexible joint; a first coupling feature disposed on the implantable medical device proximate to the flexible joint; and a second coupling feature disposed on the drug delivery module configured to mate with the first coupling feature, wherein coupling of the drug delivery module to the implantable medical device is established and maintained by an interaction between the first coupling feature and the second coupling feature.

In some embodiments, the drug delivery module comprises a rotation lock. In some embodiments, coupling of the drug delivery module to the implantable medical device is reversible. In some embodiments, coupling of the drug delivery module to the implantable medical device is irreversible.

Disclosed is a drug delivery system comprising an implantable medical device selected to deliver a first treatment for a first condition of an individual patient; a drug delivery module having at least one sidewall defining a product well; and a module insertion assembly having a top body bearing a first alignment feature, a middle body with a module recess configured to receive the drug delivery module and bearing a second alignment feature, and a base body with an implant recess configured to receive the implantable medical device and bearing a third alignment feature, wherein the middle body is configured to reversibly couple between the base body and the top body by a coaxial interaction between the first alignment feature, the second alignment feature, and the third alignment feature, and wherein compressing the middle body loaded with a drug delivery module in the module recess between the base body loaded with the implantable medical device loaded in the implant recess and the top body by a force applied to the top body under a condition wherein the first alignment feature, the second alignment feature, and the third alignment feature are coaxial causes the drug delivery module to couple to the implantable medical device.

In some embodiments, the implantable medical device is an intraocular implant. In some embodiments, the module insertion assembly is disposable.

Disclosed is a kit comprising an implantable medical device and a module insertion assembly configured to receive the implantable medical device, wherein the module insertion assembly comprises a top body, a middle body, and a base body, and wherein the module insertion assembly is configured to couple a drug delivery module to the implantable medical device.

In some embodiments, the implantable medical device is an intraocular medical device. In some embodiments, the intraocular medical device comprises an intraocular lens. In some embodiments, the module insertion assembly is disposable.

The features and advantages of the invention will be apparent to those of ordinary skill in the art from the following more particular and detailed descriptions of selected example embodiments of the disclosed system and methods, along with the accompanying drawing figures.

### BRIEF DESCRIPTION OF THE DRAWINGS

FIG. 1 is a top view of a drug-eluting intraocular implant system;

FIG. 2 is a side view of a drug-eluting intraocular implant system;

FIG. 3 is a perspective view of a drug-eluting intraocular implant system;

FIG. 4 is an enlarged perspective view of a drug delivery module of a drug-eluting intraocular implant system;

FIG. 5 is an enlarged bottom view of a drug delivery module of a drug-eluting intraocular implant system;

FIG. 6 is a top view of a drug delivery module of a drug-eluting intraocular implant system;

FIG. 7 is a side view of a drug delivery module of a drug-eluting intraocular implant system;

FIG. 8 is an enlarged perspective view of a drug delivery module of a drug-eluting intraocular implant system;

FIG. 9 is an end view of a drug delivery module of a drug-eluting intraocular implant system;

FIG. 10 is a side view of a drug delivery module of a drug-eluting intraocular implant system;

FIG. 11 is a second end view diagram of a drug delivery system assembly device;

FIG. 12 is a cutaway view of a drug delivery module of a drug-eluting intraocular implant system;

FIG. 13 is a magnified side view of a haptic arm of a drug-eluting intraocular implant system;

FIG. 14 is an exploded perspective view of a module insertion assembly of a drug-eluting intraocular implant system;

FIG. 15 is an exploded side view of a module insertion assembly of a drug-eluting intraocular implant system; and

FIG. 16 is a diagram of a method of use of a drug-eluting intraocular implant system.

### DETAILED DESCRIPTION

Various example embodiments of a drug-eluting implantable medical device systems are discussed herein. The intraocular drug delivery system 100 includes an intraocular replacement lens 102 (“IOL”) fitted with a first coupling feature 104 to connect with a drug delivery module 110. The IOL 102 prosthetic and the drug delivery module 110 are selected separately for the individual patient depending on the (1) IOL 102 needed and (2) planned drug therapy. For example, a variety of IOL 102 replacements are available. Examples of differences between IOLs include diameter, the focal length of the IOL 102, whether the IOL 102 is monofocal or toric, and others. An IOL 102 is selected by the eye surgeon in consultation with the patient to provide the optimal IOL 102 design and desired specifications.

Similarly, there is a wide and growing range of medicaments used to treat eye disease. Accordingly, a drug delivery module 110 may be created having a specific medicament, concentration, and elution mechanism to provide timed intraocular release of the medication. The manufacture of the drug delivery module 110, which delivers a specific medicament selected from any number of pharmaceutical compounds, can be coupled with an IOL 102 specific to the patient's prescription, which is selected separately from the medicament.

Embodiments of the intraocular drug delivery system 100 incorporate a secure coupling mechanism between the IOL 102 and the drug delivery module 110 whereby the surgeon or other practitioner may easily, quickly, and reliably couple the drug delivery module 110 to the IOL 102. Such a simple and reliable mechanism for coupling a drug-eluting attachment to an implantable medical device 101 is particularly important for intraocular implants, which are relatively small compared to a surgeon's hands and where detachment within the eye is potentially more serious and difficult to correct than separation of a drug-eluting attachment from a medical device 101 implanted in other locations.

In some embodiments, the drug-eluting attachment is configured to couple to an intraocular replacement lens 102 (“IOL”), which is commonly used during cataract surgery. Cataract surgery is an extremely common procedure worldwide, with the 2021 annual worldwide case rate estimated at 20 million. Many patients receiving an IOL 102 during cataract removal have coexisting ocular morbidity, including glaucoma, age-related maculopathy, and diabetic retinopathy. Additionally, postoperative medications to prevent infection, uveitis, posterior capsular opacification, and other sequelae of IOL 102 replacement during cataract surgery are also used. It can be appreciated that many ocular conditions could be treated with a drug-eluting attachment for an IOL 102 replacement medical device 101.

### Definitions

As used herein, “implantable medical device” means any therapeutic medical device 101 designed for implantation within a target tissue of a patient. As used herein, “intraocular implant” means any therapeutic medical device designed for implantation within a tissue comprised by the eye of a patient.

As used herein, “implantation” means surgical implantation, i.e., making an incision through tissue and passing the implantable medical device, such as an intraocular implant, through the incision into a target tissue, such as an eye tissue, and then closing the incision with suture or other suitable means. Implantation includes traditional “open” surgery, minimally invasive surgery, laparoscopic surgery, endoscopic or endo-luminal surgery, and other surgical approaches known in the art for placing an implantable medical device into a target tissue of a patient without limitation.

As used herein, “immediately prior to implantation” means within a period of time shortly before surgical implantation of an implantable medical device. “Immediately prior” does not have a specific time value but refers to the period of time during a surgical procedure, i.e., in the operating room, procedure room, or patient room where the surgical procedure is taking place rather than in a remote location separate from the operating room or a time separate from the surgical procedure, such as preceding the procedure by hours, days, or longer.

As used herein, “treatment” means medical care given to a patient for an illness or an injury. Examples of treatment include procedures such as surgery, administration of a medication, and the like.

As used herein, “ocular” means pertaining to or relating to the eye.

As used herein, “medicament” means any composition, compound, drug, or other substance used for medical treatment of a disease or condition. The medicament may include an active medication and a carrier substance or composition, such as a buffer, a thickener, a composition configured to dissolve within the target tissue to release the active medication from the composition over an extended period of time (“timed-release”), or the like.

As used herein, “drug eluting” means releasing a drug from a carrier composition or material over time through the action of a solvent, such as water, interstitial tissue fluid, blood, or any other fluid within the body.

As used herein, “distal” refers to a direction away from a more central part. Further, any directional references as used herein, such as right, left, up, down, top, bottom, and the like, are intended for convenience of description and do not limit the disclosed structures to any particular positional or spatial orientation.

As used herein, “anterior” means towards the front of an anatomic structure, such as the front of the face, the front of the eye, or the front-side of the body, for example.

As used herein, “posterior” means towards the back of an anatomic structure, such as the back of the head, the back of the eye, or the back side of the body, for example. Posterior refers to a side, an aspect, or a direction away from or opposite to the anterior. Similarly, anterior refers to a side, an aspect, or a direction away from or opposite to the posterior. Anterior and posterior relate to an anatomic structure's position or location.

As used herein, “radial” or “transverse” refers to a direction orthogonal to a central longitudinal axis of a structure.

As used herein, “circumferential” or “circumferentially” refers to a curved path around the body of a structure or sub-structure in a plane orthogonal to a central longitudinal axis.

As used herein, “additional embodiment,” “another additional embodiment,” “yet another additional embodiment,” “separate additional embodiment,” and similar terms refer to different examples of embodiments of drug delivery systems and methods within the scope of the disclosures and teachings found herein and the components thereof.

As used herein, “line-of-sight” or “axis of sight” means coaxial with a line passing from anterior to posterior through the center of the pupil, center of the IOL, and onto the retina of the eye.

Details of a drug eluting attachment for an implantable medical device 101 will now be discussed with reference to several drawing figures. Although many of the embodiments are described herein for implantation within the eye, it is conceived that the concept of providing a similar drug-eluting drug delivery module 110 which may be selected separately from an implantable medical device 101 and coupled to the medical device 101 in the operating room immediately prior to implantation using a simple and reliable first coupling feature 104 is within the scope of these disclosures. The utility of such a intraocular drug delivery system 100 as disclosed herein, not merely with an IOL 102 implant but across other implantable medical devices 101, will be immediately appreciated by one of skill in the art.

### Implantable Medical Device with Drug Delivery Module

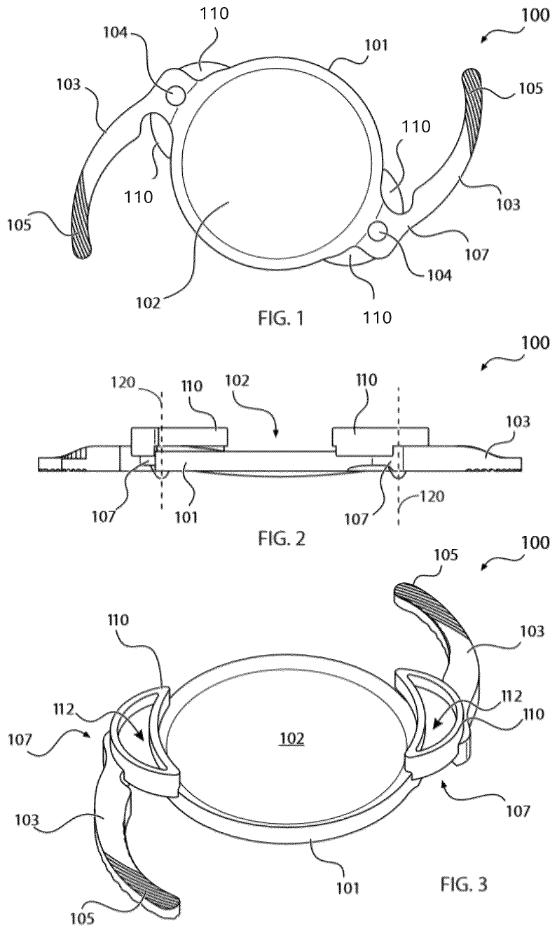

FIG. 1 is a top view of an intraocular drug delivery system 100. FIG. 1 shows an example of intraocular drug delivery system 100 with an implantable medical device 101. In this example, implantable medical device 101 is configured as an intraocular replacement lens 102 (“IOL”)—associated medical device 101 typically used in cataract surgery. Medical device 101 comprises an intraocular replacement lens 102 (IOL 102) 102 and a pair of haptic arms 103. In the example implantable medical device 101 configured for IOL 102 replacement surgery, haptic arms 103 interact with intraocular tissues to retain and center—with respect to the line-of-sight drug delivery system 100 in position within the eye. In some embodiments, medical device 101 is positioned centered beneath the pupil in the posterior chamber; however, this is not meant to be limiting. Anterior chamber placement and devices designed for scleral fixation may also be used. Haptic arms 103 present a friction grid 105 on a distal segment of each haptic arm 103 to aid in securing the system to surrounding ocular tissue, depending upon the type of IOL 102 used and the position of placement within the eye. Haptic arms 103 are each coupled to IOL 102 at a joint 107. Joint 107 allows haptic arms 103 to be placed under slight compressive tension, such as compressing a spring, to create a resilient connection such that IOL 102 remains centered beneath the pupil or otherwise with respect to the cornea and the retina of the eye.

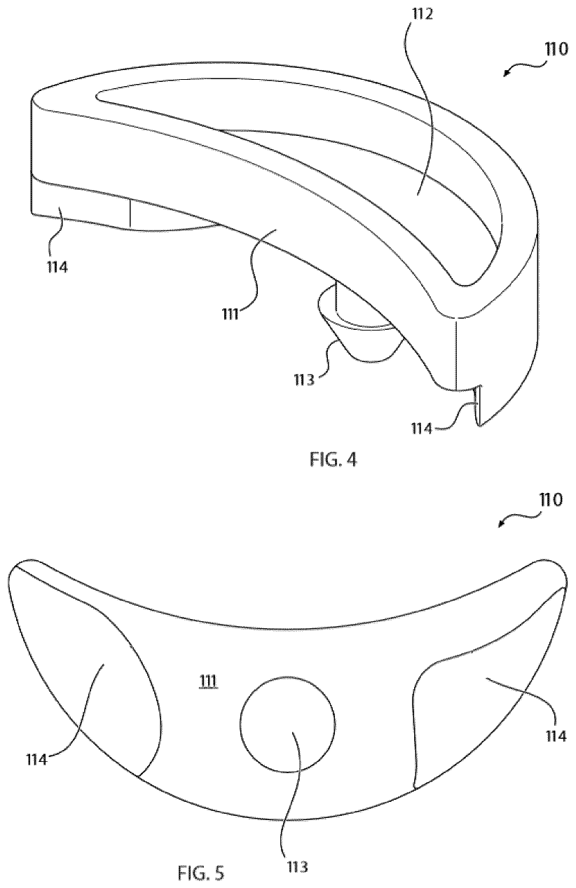

FIG. 1 additionally shows a first coupling feature 104 disposed on an external surface of medical device 101 at the location of each joint 107. In some embodiments, the external surface is a top surface, as shown in FIG. 1. In some embodiments, the external surface is a bottom surface or a side surface. First coupling feature 104 is configured to couple, i.e., “mate,” with a corresponding second coupling feature 113 (shown in FIGS. 4-5 and FIGS. 7-12 and FIG. 15) of a drug delivery module 110, discussed at length herein below. In some embodiments, the first coupling feature 104 is a bore (hole) through the full thickness of joint 107. This is not meant to be limiting, however. In some embodiments, the first coupling feature 104 is a partial thickness bore, may be normal to an external surface of joint 107, or may be angled with respect to the external surface of joint 107. Alternatively, the first coupling feature 104 may be a projection 113b or other feature protruding and extending a distance from the external surface of joint 107. The specific shape and configuration of the first coupling feature 104, in any particular embodiment, must match a corresponding complementary feature on the drug delivery module 110, as will be discussed.

FIG. 2 is a side view of an intraocular drug delivery system 100. FIG. 2 shows a drug delivery module 110 coupled to each of two haptic arms 103 proximate to joint 107. As shown in FIG. 2, drug delivery module 110 is positioned atop the junction between IOL 102 and haptic arm 103. The position of drug delivery module 110 is shown in greater detail by FIG. 3 below.

FIG. 3 is a perspective view of an intraocular drug delivery system 100. FIG. 3 shows drug delivery module 110 coupled to each haptic arm 103 at each joint 107. Note that drug delivery module 110 is shaped so as not to overlay any light-transmitting portion of IOL 102. This is important to prevent drug delivery module 110 from interfering with vision. This design, shape, and overall configuration of intraocular drug delivery system 100 described herein is specific to medical device 101 configured as an implantable IOL 102. Alternate shapes and configurations would be used in an embodiment of intraocular drug delivery system 100 utilizing other alternative implantable medical devices 101.

Drug delivery module 110 comprises a product well 112. Product well 112 is configured to retain a medicament for delivery into tissues surrounding the site of implantation of medical device 101. In some embodiments, product well 112 is open, such as the example embodiment shown in FIG. 3 and throughout the several drawing figures. In some embodiments, product well 112 is fenestrated. In some embodiments, product well 112 comprises a permeable membrane constraining the medicament such that the medicament diffuses through the permeable membrane at a predetermined rate consistent with the chemical composition of the medicament and conditions at the implantation anatomic site, such as pH or osmolality, for example. In some embodiments, the medicament is distributed throughout a carrier. In some embodiments, the carrier is a biopolymer. Examples of biopolymers and related drug delivery systems include hydrogels, cellulose-based compositions, other protein-based compositions, and the like are provided by “Recent developments in natural biopolymer-based drug delivery systems” by Fazel, T., et al., Royal Society of Chemistry Advances vol. 13 pp. 23087-121 (2023), which is incorporated in its entirety herein by reference. Various other controlled or time-release mechanisms for the medicament packaged within product well 112 are contemplated and within the scope of these disclosures.

Although many different configurations of implantable medical device 101 and drug delivery module 110 are possible, a key element of intraocular drug delivery system 100 is to allow a practitioner to select a particular configuration of medical device 101 separately from a medicament and match the medical device 101 with drug delivery module 110 bearing a specific drug delivery composition containing a specific medicament. Medical device 101 is selected according to the implantation site, size of the patient, and other anatomic or physical characteristics specific to the patient independent of the disease condition being treated. Drug delivery module 110 is selected according to the disease condition being treated and is not dependent on the physical characteristics of an individual patient. The desired medicament is packaged in a drug delivery composition contained within product well 112. Moreover, medical device 101 and drug delivery module 110 are packaged separately. They may have separate expiration dates, and medical device 101 and drug delivery module 110 may be manufactured separately by different manufacturers at different times in separate locations if needed. The third alignment features 208, additionally, allows the surgeon or other practitioner to select and couple medical device 101 and drug delivery module 110 immediately prior to implantation, such as in the operating room, for example. In some cases, the surgeon may not be certain of the size or specific model of the implantable medical device 101 to be implanted until the implantation site is surgically exposed and examined by the surgeon in the operating room. Consequently, the third alignment features 208 listed herein add tremendous versatility to medical device 101 and drug manufacturing processes, facility inventory, procedure planning, and other treatment aspects specific to an individual patient, along with other advantages.

FIG. 4 is an enlarged perspective view of a drug delivery module 110 of an intraocular drug delivery system 100. FIG. 4 shows drug delivery module 110 having a body 111 defining the product well 112. Second coupling feature 113 is shown projecting from a surface of body 111 opposite product well 112 in this and some other embodiments.

FIG. 5 is an enlarged bottom view of a drug delivery module 110 of an intraocular drug delivery system 100. FIG. 5 shows the second coupling feature 113 generally centered on a bottom surface of the body 111. The second coupling feature 113 of drug delivery module 110 is configured to interact with the first coupling feature 104 of implantable medical device 101, coupling drug delivery module 110 to medical device 101. In the example shown in the several drawing figures and some embodiments, second coupling feature 113 is a mushroom-shape projecting “button” such that second coupling feature 113 may be pushed through a corresponding aperture comprised by first coupling feature 104, locking drug delivery module 110 to implantable medical device 101. In some embodiments of intraocular drug delivery system 100, employing this configuration of first coupling feature 104 and second coupling feature 113, are partially or completely shaped from a deformable elastic material such that the mushroom-shaped head 113a may be sufficiently deformed to push through the bore forming first coupling feature 104. In some embodiments, the mating of the first coupling feature 104 and the second coupling feature 113 is reversible. This is desirable and useful for embodiments of intraocular drug delivery system 100 wherein drug delivery module 110 must be replaced one or more times following implantation of a medical device 101 when a therapeutic medicament residing in product well 112 becomes depleted and must be replaced. In some embodiments, the mating of first coupling feature 104 and second coupling feature 113 is irreversible such that drug delivery module 110 and implantable medical device 101 are permanently inseparable after coupling together. This is desirable and useful for embodiments of intraocular drug delivery system 100 wherein replacement of drug delivery module 110 is associated with unacceptably high risk or complications or is otherwise impractical because of the anatomic location wherein implantable medical device 101 is positioned.

The “button-in-hole” interaction between second coupling feature 113 and first coupling feature 104, as shown in several drawing figures, is provided by example only. Other coupling mechanisms are possible in some embodiments. Some non-limiting examples of alternative designs of other coupling mechanisms include interacting tabs, clips, snaps, and the like, as presented by first coupling feature 104 and second coupling feature 113.

FIGS. 4-5 also shows an example embodiment of body 111 comprising a rotation lock 114. In some embodiments, a rotation lock 114 resists the rotation of drug delivery module 110 on the implantable medical device 101. Rotation lock 114, in some embodiments, interacts with a corresponding first coupling feature 104, such as a lip, a rim, or a complementary shelf (not shown in the drawing figures) presented by implantable medical device 101. A person of skill will see the importance, in some embodiments of intraocular drug delivery system 100 wherein drug delivery module 110 is not rotationally symmetrical, to prevent rotation of drug delivery module 110 about an axis 120 defined by first and second coupling features 104 and 113 from causing undesired interference with physiologic functions at the implantation site. For example, such rotational stability is particularly important when implantable medical device 101 is an IOL 102 medical device 101 because with any rotation of a crescent-shaped drug delivery module 110 over IOL 102, drug delivery module 110 will cross into the line-of-sight and enter a light path, interfering with the recipient patient's vision.

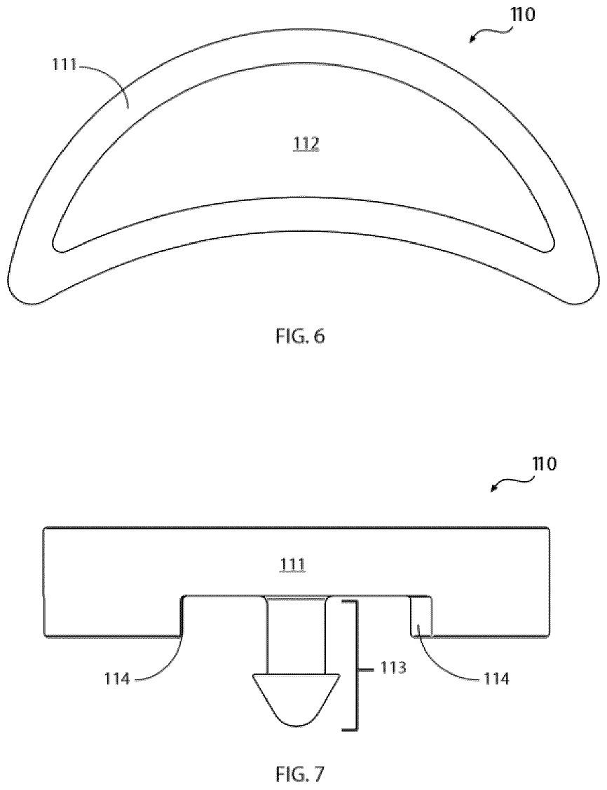

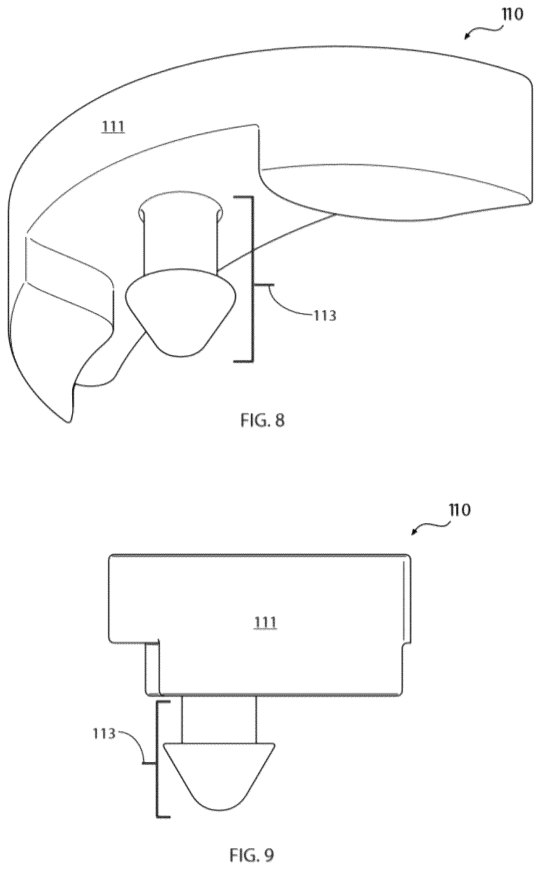

FIG. 6 is a top view of a drug delivery module 110 of an intraocular drug delivery system 100. FIG. 7 is a side view of a drug delivery module 110 of an intraocular drug delivery system 100. FIG. 8 is an enlarged perspective view of a drug delivery module 110 of an intraocular drug delivery system 100. FIG. 9 is an end view of a drug delivery module 110 of an intraocular drug delivery system 100. FIGS. 6-9 show several views depicting the third alignment features 208 of drug delivery module 110, including the second coupling feature 113, rotation lock 114, and product well 112. The embodiment of drug delivery module 110 shown in the drawing figures is specific to intraocular drug delivery system 100 incorporating an IOL implantable medical device 101; however, other configurations of drug delivery module 110 are contemplated configured to couple to other implantable medical devices within intraocular drug delivery system 100.

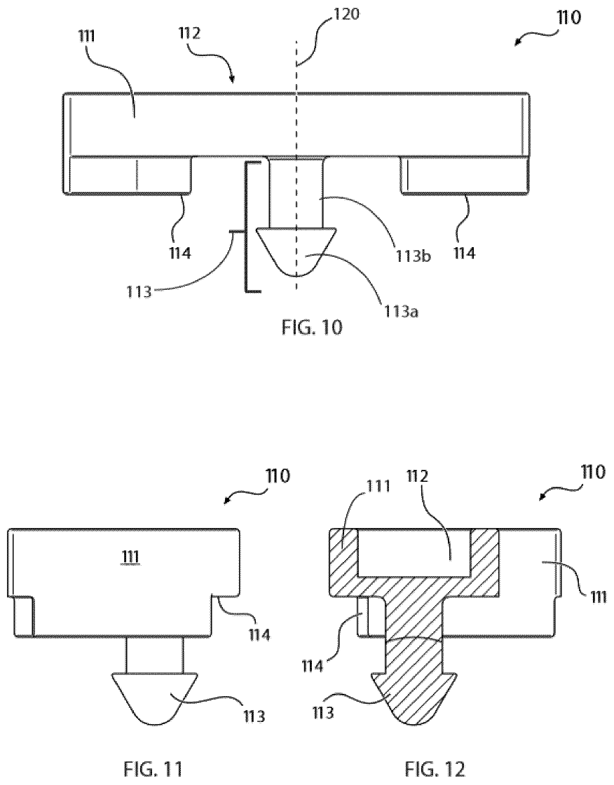

FIG. 10 is a side view of a drug delivery module 110 for an intraocular drug delivery system 100. FIG. 10 shows drug delivery module 110 having body 111 with two rotation locks 114 and a second coupling feature 113. As shown in FIG. 10, the second coupling feature 113 projects from drug delivery module 110. In some embodiments, including the example shown in FIG. 10, the second coupling feature 113 comprises a head 113a and a projection 113b. Head 113a has a generally frustoconical shape and is formed from a resilient material to pass through the first coupling feature 104, locking implantable medical device 101 to drug delivery module 110, in some embodiments. In some embodiments, head 113a is formed from a substantially rigid material, and first coupling feature 104 is formed from or lined by a layer of substantially deformable, resilient materials to allow passage of head 113a through first coupling feature 104. Projection 113b acts offset head 113a from body to allow full transit of head 113a through the first coupling feature 104, in some embodiment. Other configurations of first coupling feature 104 and second coupling feature 113, as well as the form of interaction between first coupling feature 104 and second coupling feature 113, are contemplated by the disclosures herein. FIG. 10 also shows the axis of rotation passing through a central long axis 120 of the second coupling feature 113.

FIG. 11 is a second end view diagram of an intraocular drug delivery system 100 assembly device 200, and FIG. 12 is a cutaway view of a drug delivery module 110 of an intraocular drug delivery system 100. FIGS. 11-12 show additional details of some embodiments of drug delivery module 110. In some embodiments, product well 112 and second coupling feature 113 are formed as a unitary body with body 111. In some embodiments, the second coupling feature 113 is formed separately from body 111 and later bonded to body 111 by use of adhesives, heat-welding, or other suitable techniques.

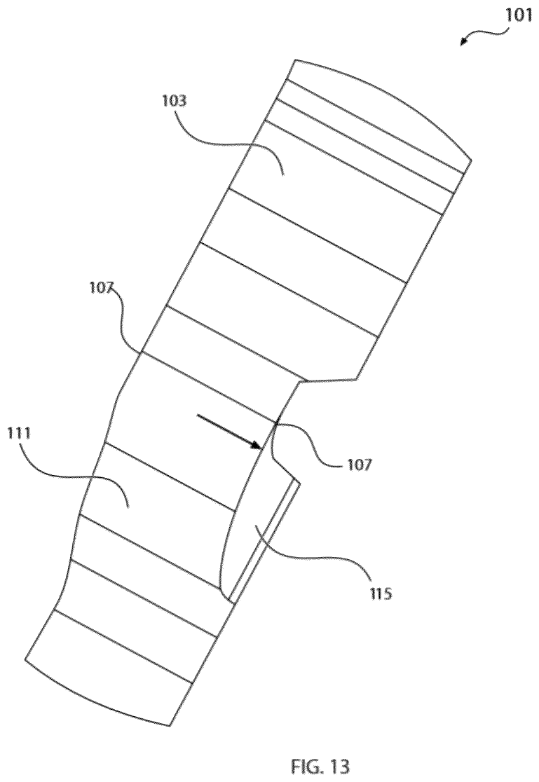

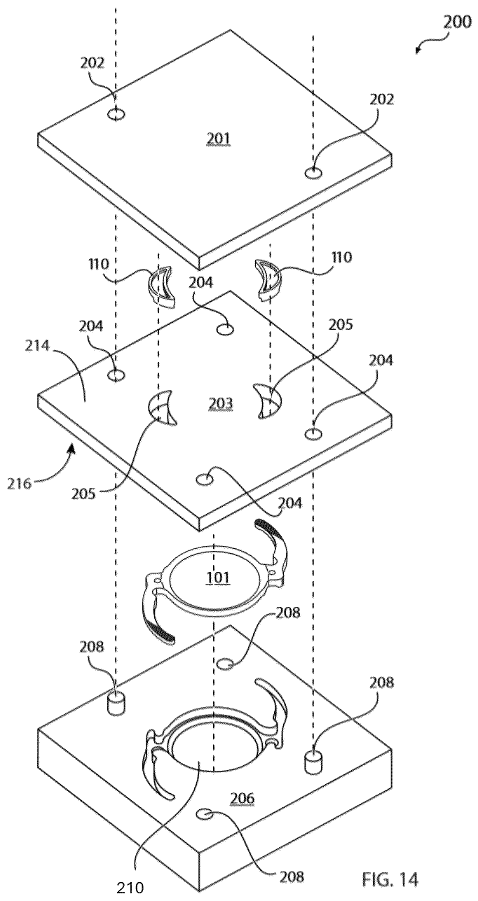

FIG. 13 is a magnified view of the junction of a haptic arm 103 with an intraocular replacement lens 102 of an intraocular drug delivery system 100. FIG. 13 shows an example of an implantable medical device 101 consistent with intraocular drug delivery system 100 comprising an IOL 102. Haptic arm 103 is coupled to body 111 at a joint 107. As discussed herein, joint 107 facilitates the fixation and centering of implantable medical device 101 configured as an intraocular implant within the eye. Also shown in FIG. 13 is a corresponding feature 115. In embodiments of intraocular drug delivery system 100 wherein drug delivery module 110 is not rotationally symmetrical or otherwise must not rotate with respect to implantable medical device 101, corresponding feature 115 interacts with rotation lock 114 of drug delivery module 110 to resist rotation of drug delivery module 110 about axis 120 passing through first coupling feature 104 and second coupling feature 113.

### Module Insertion Assembly

Intraocular drug delivery system 100 includes, in some embodiments, an assembly device 200 configured to enable or assist with the coupling of the implantable medical device 101 and drug delivery module 110. The use of an assembly device 200 is desirable in some embodiments of the intraocular drug delivery system 100, such as for use with implantable medical device 101, which is configured for use with a drug delivery module 110, measuring less than 10 millimeters in greatest dimension. Drug delivery modules 110 of this size and smaller can be difficult to manipulate with the fingers, leading to lengthy times spent coupling the drug delivery module 110 to implantable medical device 101. Saving time in the operating room is of paramount importance for many reasons known to those of skill in the art of surgically implanted medical devices. Coupling an implantable medical device 101 with a drug delivery module 110 requires a controlled application of force to approximate a first coupling feature 104 and a second coupling feature 113 along a properly aligned axis 120. Improper alignment between the first and second coupling features 104 and 113 increased the risk of breakage of delicate components, such as those forming an implantable drug-eluting intraocular medical device 101. Also, manipulating small objects increases the risk of dropping medical device components on the operating room floor, resulting in the need to discard a contaminated or damaged drug delivery module 110 or the implantable medical device 101.

Consequently, disclosure of the assembly device 200 is provided, at least to (1) reduce operating room time needed for assembly of an intraocular drug delivery system 100; (2) reducing the risk of damaging intraocular drug delivery system 100 components during assembly; and (3) minimizing the risk of ruining potentially expensive components of limited availability through contamination or breakage.

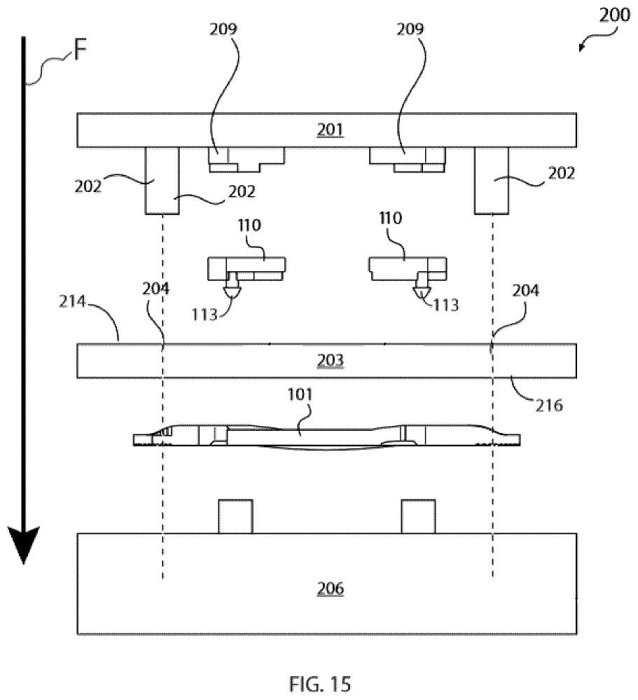

FIG. 14 shows an exploded perspective view diagram of an assembly device 200 for an intraocular drug delivery system 100. FIG. 15 is an exploded side view of an assembly device 200 of an intraocular drug delivery system 100. FIGS. 14-15 show an assembly device 200 of intraocular drug delivery system 100. The assembly device 200, in some embodiments, is formed by aligned, stacked components comprising a base body 206, a middle body 203, and a top body 201. The base body 206, middle body 203, and top body 201 fit together in alignment and are reversibly assembled and disassembled through coaxial alignment of one more series of complementary first alignment features 202, as shown in FIGS. 14-15. In some embodiments, a first alignment feature 202 is disposed on top body 201, a second alignment feature 204 is disposed on the middle body 203, and a third alignment feature 208 is disposed on the base body 206. In some embodiments, one of either first alignment feature 202 or third alignment feature 208 is formed as a protrusion configured to reversibly couple with a corresponding first coupling feature 104, such as an aperture sized to accommodate the protrusion, forming the other of either first alignment feature 202 or third alignment feature 208.

The base body 206 includes a device receiver 210. Device receiver 210 is, essentially, a “cutout” sized and shaped large enough to easily receive the implantable medical device 101, such as medical device 101, but not so large as to allow movement of a first coupling feature 104 born by medical device 101 with a second coupling feature 113 displayed by a drug delivery module 110, such as drug delivery module 110. In some embodiments, it is used with an IOL 102 implantable medical device 101 and as shown in FIG. 10, device receiver 210 is dimensioned to receive and constrain an IOL 102 coupled to haptic arms 103. This is not meant to be limiting. A medical device 101 can be dimensioned to receive and constrain other implantable medical devices for coupling to drug delivery modules 110 to form an intraocular drug delivery system 100, in some other embodiments, without limitation.

Middle body 203, in some embodiments, comprises one or more module recesses 205. Middle body 203 comprises a first surface 214 and a second surface 216 opposite the first surface 214. When the top body 201, middle body 203, and base body 206 are assembled, the first surface 214 faces the top body 201, and the second surface 216 faces the base body 206. Middle body 203 additionally comprises a second alignment feature 204. In some embodiments, including the example shown in FIGS. 14-15, middle second alignment feature 204 is an aperture configured to receive a protrusion forming either first alignment feature 202 or third alignment feature 208. Alignment features 202, 204, and 208 function to align corresponding sections of top body 201, middle body 203, and base body 206 so as to maintain first coupling feature 104 of implantable medical device 101 and second coupling feature 113 of drug delivery module 110 aligned along axis 120.

FIG. 14 also shows a pair of module recesses 205 on middle body 203. Module recess 205 is configured to receive and constrain device receiver 210 for coupling with implantable medical device 101, as will be discussed shortly. Middle body 203 is configured to be sandwiched between base body 206 and top body 201, as shown in FIGS. 14-15.

Top body 201 comprises one or more implant presses 209. Each implant press 209 is formed as a protrusion having a profile similar to that presented by device receiver 210, as shown in FIG. 15 and in some embodiments. When assembly device 200 is fitted with an implantable medical device 101 and a drug delivery module 110 and fully assembled or “stacked” and squeezed together, each implant press 209 transmits a force F applied by the user to the loaded and fully assembled module insertion assembly device 200 upon each corresponding drug delivery module 110, urging together each corresponding first coupling feature 104 and second coupling feature 113.

FIG. 15 shows middle body 203 atop base body 206. Middle body 203 and base body 206 are seen in FIG. 15 to be substantially aligned by the interaction of a corresponding plurality of alignment features 202, 204, and 208. FIG. 11 also shows a pair of implant presses 209 disposed on an under-surface of top body 201. Each implant press 209 is formed as a protruding first coupling feature 104 of the shape and slightly smaller size as a cross-section of drug delivery module 110. The implantable medical device 101 is received in a module recess 205 within base body 206, forming a device receiver 210. Each module recess 205 of middle body 203, as shown in the partially assembled assembly device 200 of FIG. 11, is positioned such that module recess 205 overlays the first coupling feature 104 of an implantable medical device 101, wherein medical device 101 rests within device receiver 210. In this manner, drug delivery module 110 can be placed within module recess 205 such that the second coupling feature 113 contacts the first coupling feature 104. The top body 201 is then positioned atop the assembly and aligned by the interaction of first alignment feature 202 and locking third alignment feature 208 with implant presses 209 contacting a corresponding plurality of drug delivery modules 110. With all sections of assembly device 200 held in alignment and loaded with medical device 101 and drug delivery module 110, a force “F” (shown in FIG. 15) is applied to push top body 201 and base body 206 toward one another against the middle body 203, thereby forcing first coupling feature 104 and second coupling feature 113 to lock together and “mate” while constraining drug delivery module 110 and implantable medical device 101 in alignment. In some embodiments, including the example embodiment shown in FIGS. 14-15, this maneuver locks a drug delivery module 110 onto the haptic arm body 111 juncture of an implantable intraocular medical device 101 proximate to the joint 107 at the mated first and second coupling features 104 and 113, respectively.

FIG. 14 shows that third alignment feature 208 is configured as a protrusion in some embodiments and a module recess 205 in some embodiments. In some embodiments, including the example embodiment shown in FIG. 14, the base body 206 comprises a plurality of third alignment features 208, one or more third alignment features 208 formed as a protrusion, and one or more third alignment features 208 formed as a module recess 205. Similarly, top body 201, in some embodiments, comprises a corresponding number of one or more first alignment features 202 to the number of third alignment features 208 with a corresponding configuration wherein a recessed first alignment feature 202 receives a protrusion third alignment feature 208 and a recess third alignment feature 208 receives a protrusion first alignment feature 202. It is conceived and within the scope of these disclosures that second alignment feature(s) 204 presented by middle body 203 are formed, in some embodiments (not shown in the several drawing figures) as protrusions extending from first surface 214 of middle body 203 and/or second surface 216 of middle body 203 and are received by corresponding recesses forming first alignment feature 202 and third alignment feature 208 respectively.

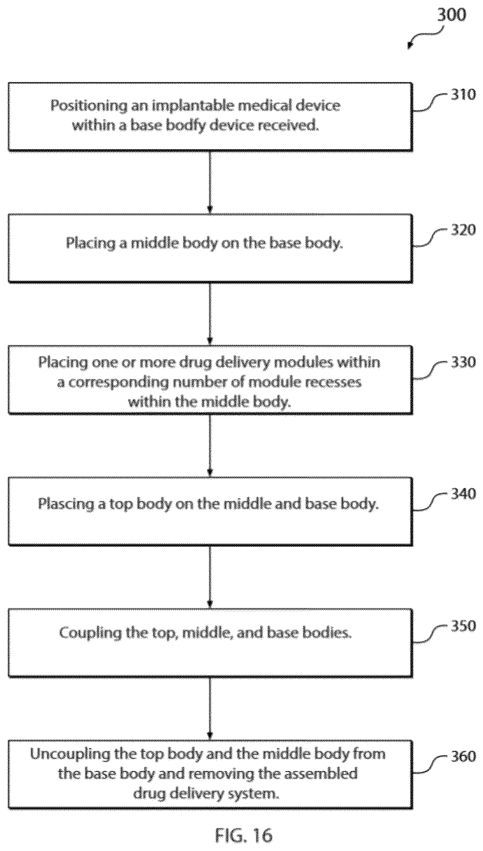

FIG. 16 is a diagram of a method 300 of assembling an intraocular drug delivery system 100. FIG. 16 shows a method 300 of forming an intraocular drug delivery system 100 for an implantable medical device 101 comprising a positioning step 310, a first placing step 320, a second placing step 330, a third placing step 340, a coupling step 350, and an uncoupling step 360.

In some embodiments, positioning step 310 comprises positioning an implantable medical device 101 within a base body device receiver 210. In some embodiments, the implantable medical device 101 is an IOL 102. In some embodiments, the IOL 102 has haptic arms 103.

In some embodiments, the first placing step 320 comprises placing a middle body 203 on the base body 206. In some embodiments, first placing step 320 includes threading one or more second alignment features 204 of the middle body 203 onto a corresponding one or more locking first alignment features 202 of the base body 206 such that the middle body 203 is very closely aligned in position atop of the base body 206. “Aligned in position” means that when the middle body 203 is coupled to the base body 206, one or more module recesses 205 of the middle body 203 are maintained in a position directly above corresponding positions of the base body device receiver 210 by an interaction between the second alignment feature(s) 204 and third alignment feature(s) 208 such that each first coupling feature 104 of the implantable medical device 101 is aligned over each corresponding second coupling feature 113 of a drug delivery module 110 loaded into each module recess 205 of the middle body 203.

In some embodiments, second placing step 330 comprises placing one or more drug delivery modules 110 within a corresponding number of module recesses 205 within the middle body 203 after the middle body 203 has been positioned atop and aligned with the base body 206. The drug delivery modules 110 are placed such that a second coupling feature 113 of the drug delivery module 110 resting within the module recess 205 contacts a corresponding first coupling feature 104 of the implantable medical device 101 constrained within the device receiver 210 of the base body 206 and held in place by the middle body 203.

In some embodiments, the third placing step 340 comprises placing a top body 201 on the middle and base body 206. Third placing step 340 includes positioning a first alignment feature 202 of the top body 201 onto the locking third alignment feature 208 of the base body 206 such that the top body 201 is very closely aligned atop the middle body 203 wherein one or more implant presses 209 are positioned in contact with the corresponding number of drug delivery modules 110.

In some embodiments, coupling step 350 comprises coupling the top, middle and base bodies. In some embodiments, this is accomplished simply by pushing the top body 201 and the base body 206 together, i.e., by applying a downward force to the top body 201 or by compressing together the top, middle, and base bodies such that the one or more drug delivery modules 110 loaded into the middle body 203 are snap-locked onto the implantable medical device 101 loaded into the base body 206 by a mating of the corresponding first and second coupling features 104 and 113 of the implantable medical device 101 and the drug delivery module(s) 110.

In some embodiments, uncoupling step 360 comprises uncoupling the top body 201 and the middle body 203 from the base body 206 and removing the assembled intraocular drug delivery system 100.

In some embodiments, assembly device 200 is packaged together with one or more drug delivery modules 110 as part of a kit. In some embodiments, the assembly device 200 is provided pre-packaged in sterile packaging. In some embodiments, assembly device 200 is clean-packaged and later sterilized by the end-user. In some embodiments, assembly device 200 is disposable. In some embodiments, assembly device 200 is re-usable.

Several embodiments of an intraocular drug delivery system 100 for an implantable medical device 101 have been presented. The intraocular drug delivery system 100 creates versatility by choosing a drug delivery module 110 containing a specific medicament for coupling with a specific size and configuration of an implantable medical device 101 for use in locations wherein many medical device 101 sizes and configurations are needed, and many therapeutic medicinal compositions may be used to increase therapeutic efficacy by eliminating patient self-dosing and associated problems with compliance. This is particularly useful in the eye, where a drug delivery module 110 may be selected and easily, securely coupled to an IOL 102 in the operating room immediately prior to implantation. Other combinations of implantable medical devices fitted with a first coupling feature 104 for securely coupling to a drug delivery module 110 are contemplated by such an intraocular drug delivery system 100. In some embodiments, the intraocular drug delivery system 100 for an implantable medical device 101 includes an assembly device 200 to decrease operating room time, reduce breakage and contamination, and facilitate assembly and use of the implantable medical device 101 coupled to a drug delivery module 110.

The embodiments and examples set forth herein were presented in order to best explain the present invention and its practical application and to thereby enable those of ordinary skill in the art to make and use the invention. However, those of ordinary skill in the art will recognize that the foregoing description and examples have been presented for the purpose of illustration and example. The description as set forth is not intended to be exhaustive or to limit the invention to the precise form disclosed. Many modifications and variations are possible in light of the teachings herein above.

What is claimed is:

1. A method comprising:
providing a medical device, a drug delivery module, and an assembly device; wherein the medical device comprises a first coupling feature; the drug delivery module comprises a second coupling feature and a product well; and the assembly device comprises a top body comprising an implant press, middle body defining a module recess, and a base body defining a device receiver;
placing the medical device in the device receiver and the drug delivery module in the module recess;
aligning the top body, the middle body, and the base body;
applying a force to the top body, wherein the force is sufficient for the implant press to cause the first coupling feature and the second coupling feature to couple to each other.

2. The method of claim 1, further comprising selecting the drug delivery module, wherein the drug delivery module comprises drug-eluting medication.

3. The method of claim 1, wherein the drug delivery module is configured to fit at least partially in the device receiver.

4. The method of claim 1, wherein the drug delivery module is configured to be secured in the device receiver by an interference fit.

5. The method of claim 1, wherein the applying the force to the top body further comprises causing the implant press to at least partially enter the device receiver.

6. The method of claim 1, further comprising removing the top body and the middle body from the bottom; and retrieving a constructed drug delivery system.

7. The method of claim 6, wherein the constructed drug delivery system is an intraocular drug delivery system.

8. The method of claim 1, further comprising inserting a medication into the drug delivery module.

9. The method of claim 1, wherein the applying the force to the top body comprises manually applying the force.

10. The method of claim 1, wherein the applying the force to the top body comprises a user placing a hand on the top body and pressing on the top body.

11. The method of claim 1, wherein the aligning the top body, the middle body, and the base body comprises alignment features of the top body, the middle body, and/or the base body with each other.

12. The method of claim 11, wherein the alignment features comprise projections and holes.

13. The method of claim 1, wherein the first coupling feature defines a hole and the second coupling feature comprises a projection and a head.

14. The method of claim 13, wherein the applying the force to the top body comprises forcing the head through the hole.

15. The method of claim 1, further comprising selecting the drug delivery module based on a diagnosed condition of a patient.

16. The method of claim 15, wherein the selecting the drug delivery module comprises selecting from multiple available drug delivery modules, each with varying sizes, shapes, and/or drug-eluting medication.

17. A method comprising:
providing a medical device, a drug delivery module, and an assembly device; wherein the medical device comprises a first coupling feature; the drug delivery module comprises a second coupling feature and a product well; and the assembly device comprises a top body, middle body defining a module recess, and a base body defining a device receiver;
placing the medical device in the device receiver and the drug delivery module in the module recess;
aligning the top body, the middle body, and the base body;
applying force to the top body, wherein the force is sufficient to cause the first coupling feature and the second coupling feature to couple to each other.

18. A method comprising:
constructing a drug delivery system, wherein the constructing the drug delivery system comprises:
providing a medical device, a drug delivery module, and an assembly device;
wherein the medical device comprises a first coupling feature; the drug delivery module comprises a second coupling feature and a product well; and the assembly device comprises a top body, middle body defining a module recess, and a base body defining a device receiver;
placing the medical device in the device receiver and the drug delivery module in the module recess;
aligning the top body, the middle body, and the base body; and
applying force to the top body, wherein the force is sufficient to cause the first coupling feature and the second coupling feature to couple to each other.
